(19) United States
(12) Patent Application Publication
(10) Pub. No.: US 2008/0221111 A1
(43) Pub. Date: Sep. 11, 2008

(54) Combination of a Pd4 Inhibitor and a Tetrahydrobiopterin Derivative

(75) Inventors: Christian Hesslinger, Zoznegg (DE); Christian Schudt, Konstanz (DE); Degenhard Marx, Moos (DE); Clemens Braun, Konstanz (DE)

Correspondence Address: NATH & ASSOCIATES PLLC, 112 South West Street, Alexandria, VA, 22314, US

(73) Assignee: NYCOMED GmbH, Konstanz (DE)

(21) Appl. No.: 11/919,742

(22) PCT Filed: May 8, 2006
(86) PCT No.: PCT/EP2006/062117

§ 371 Date: Dec. 19, 2007

(30) Foreign Application Priority Data
May 11, 2005 (EP) 05103938.6

(51) Int. Cl. A61K 31/4985; A61K 31/44; A61P 11/00; A61P 21/00

(52) U.S. Cl. 514/249; 514/352

(57) ABSTRACT

The invention describes the use of a PDE4 inhibitor in combination with BH4 or a BH4 derivative for the prevention and/or treatment of respiratory diseases.

### FIELD OF THE INVENTION

The invention relates to a combination of a PDE4 inhibitor and a tetrahydrobiopterin derivative. Furthermore, the invention relates to the use of this new combination for the prevention and/or treatment of respiratory diseases.

### BACKGROUND OF THE INVENTION

The reduction of endothelium-dependent vasodilatation is mainly induced by a decreased bioavailability of the endothelium-dependent vasodilator nitric oxide (NO) and an increase in the activity of toxic oxygen free radicals such as superoxide anions acting as vasoconstrictors.

It is known from prior art that Nitric Oxide Synthases [NOS: nNOS(NOS1), iNOS(NOS2) and eNOS (NOS3)] produce both NO and superoxide anions. The key in the net outcome of NO production by NOS seems to be the presence of (6R)-L-erythro-5,6,7,8-tetrahydrobiopterin (hereinafter referred to as “BH4”).

BH4 is an essential co-factor of NOS as it influences the rate of NO vs. superoxide production by NOS [Werner-Felmayer G et al. (2002) Current Drug Metabolism 3: 159]. In conditions when BH4 is reduced, a NOS produces superoxide anions instead of NO [Vasquez-Vivar et al. (1998) PNAS 95: 9220]. NO is rapidly deactivated by superoxide anions resulting in the formation of vasotoxic peroxynitrite (ONOO⁻). In the presence of the toxic oxide radicals, i.e. superoxide anion and ONOO⁻, BH4 is degraded to BH2 (L-erythro-7,8-dihydro-biopterin). BH2 does not act as co-factor for NOS and negatively influences NOS activity [Landmesser et al. J Clin Invest (2003) 111: 1201]. In parallel, ONOO⁻ uncouples NOS so that NOS produces superoxide anion instead of NO. In the vasculature, NO plays a central role in vasodilatation whereas superoxide leads to vasoconstriction. The degradation of BH4 and the uncoupling of NOS and the resulting reduced NO concentration in the endothelium lead to vasoconstriction and finally to pulmonary hypertension.

It is known from prior art that BH4 plays a key role in a number of biological processes and pathological states associated with neurotransmitter formation, vasorelaxation, and immune response [Werner-Felmayer G et al. (2002) Current Drug Metabolism 3: 159]. As an example deficient production of BH4 is associated with “atypical” phenylketonuria [Werner-Felmayer G et al. (2002) Current Drug Metabolism 3: 159] and provides the basis for endothelial dysfunction in atherosclerosis, diabetes, hypercholesterolaemia and smoking [Tiefenbacher et al. (2000) Circulation 102: 2172, Shinozaki et al (2003) J Pharmacol Sci 91: 187, Fukuda et al (2002) Heart 87: 264, Heitzer et al (2000) Circulation 86: e36].

It is also known in the art that BH4 improves endothelial dysfunction and thereby increases the availability of NO and decreases the presence of toxic radicals. BH4 has a beneficial effect for endothelial function caused by its cofactor role for NOS [Werner-Felmayer G et al. (2002) Current Drug Metabolism 3: 159].

As known from prior art, BH4 and its use as a medicament has been associated with several diseases. According to Ueda et al. [Ueda S et al. (2000) J. Am. Coll. Cardiol. 35:71], BH4 can improve endothelial-dependent vasodilatation in chronic smokers. According to Mayer W. et al. [Mayer W. et al. (2000) J. Cardiovasc. Pharmacol. 35: 173] coronary flow responses in humans are significantly improved by application of BH4. WO9532203 refers to the use of NOS-inhibitory pteridine derivatives (“anti-pterines”) for the treatment of diseases caused by increased NO levels. In particular, in accordance with WO9532203, inhibitory pteridine derivatives are described for prevention and treatment of pathological blood pressure decrease, colitis ulcerosa, myocardial infarction, transplant rejection, Morbus Alzheimer, epilepsy and migraine. EP0908182 refers to pharmaceutical compositions comprising BH4 or derivatives thereof for prevention and/or treating of diseases associated with dysfunction of NOS. WO0156551 discloses the use of BH4 and cGMP analogues for the treatment of respiratory diseases such as pneumonia and asthma. EP0209689 refers to the use of tetrahydrobiopterins in the preparation of a medicament for the treatment of infantile autism. WO2005041975 discloses the use of BH4 or derivatives thereof for the treatment of COPD; also disclosed in this international patent application is the use of a combination of BH4 or derivatives thereof with arginine or derivatives thereof for the treatment of COPD.

Cyclic nucleotide phosphodiesterase (PDE) inhibitors, particularly inhibitors of type 4 (PDE4), are useful in the treatment of a variety of allergic and inflammatory diseases, for example in respiratory diseases such as asthma and chronic obstructive pulmonary disease (COPD). Lipworth B reviews in Lancet 2005, Vol 365, pp 176-175 the use of PDE4 inhibitors, in particular Roflumilast and Cilomilast, for the treatment of asthma and COPD. In Drugs in R & D, Vol 5, No 3, 2004, pp 176-181 the PDE4 inhibitor Roflumilast is reviewed.

The PDE4 inhibitors, which are part of the new combination according to the invention are disclosed in the international patent application WO9501338.

It would be desirable to provide combinations that take advantage of the different therapeutic pathways of BH4 or derivatives thereof on the one hand side and a PDE4 inhibitor on the other hand side to treat a variety of respiratory diseases, in particular COPD.

### DESCRIPTION OF THE INVENTION

Surprisingly, it has been found that the combination of Roflumilast with BH4 has advantageous effects in the prevention and/or treatment of respiratory diseases with an underlying partial and global respiratory failure; the combination is particularly beneficial in the prevention and/or treatment of COPD.

Therefore, according to a first aspect of the invention there is provided a combination product comprising a pharmaceutical formulation including an amount of a first therapeutic compound selected from the group consisting of Roflumilast, a pharmaceutically acceptable salt of Roflumilast, Roflumilast-N-oxide and a pharmaceutically-acceptable salt of Roflumilast-N-oxide, an amount of a second therapeutic compound selected from the group consisting of BH4, a pharmaceutically acceptable salt of BH4, a BH4 derivative and a pharmaceutically acceptable salt of a BH4 derivative, wherein the first amount and the second amount together comprise a therapeutically effective amount for the prevention and/or treatment of respiratory diseases, and optionally pharmaceutically acceptable adjuvants, diluents and/or carriers.

The combination product according to the invention provides for the administration of a first therapeutic compound selected from the group consisting of Roflumilast, a pharmaceutically acceptable salt of Roflumilast, Roflumilast-N-oxide and a pharmaceutically-acceptable salt of Roflumilast-N-oxide in conjunction with a second therapeutic compound selected from the group consisting of BH4, a pharmaceutically acceptable salt of BH4, a BH4 derivative and a pharmaceutically acceptable salt of a BH4 derivative, and may thus be presented either as combined preparation (i.e. presented as a single formulation including the first and second therapeutic compound) or may be presented as separate formulations, wherein at least one of those formulations comprises the first therapeutic compound and at least one comprises the second therapeutic compound.

Thus, there is further provided:

A combination product comprising: (A) an amount of a first therapeutic compound selected from the group consisting of Roflumilast, a pharmaceutically acceptable salt of Roflumilast, Roflumilast-N-oxide and a pharmaceutically-acceptable salt of Roflumilast-N-oxide; and (B) an amount of a second therapeutic compound selected from the group consisting of BH4, a pharmaceutically acceptable salt of BH4, a BH4 derivative and a pharmaceutically acceptable salt of a BH4 derivative, wherein the first amount and the second amount together comprise a therapeutically effective amount for the prevention and/or treatment of respiratory diseases and wherein each of components (A) and (B) is optionally formulated in admixture with pharmaceutically acceptable adjuvants, diluents and/or carriers.

A kit of parts comprising components: (a) a pharmaceutical formulation including an amount of a first therapeutic compound selected from the group consisting of Roflumilast, a pharmaceutically acceptable salt of Roflumilast, Roflumilast-N-oxide and a pharmaceutically acceptable salt of Roflumilast-N-oxide, optionally in admixture with pharmaceutically acceptable adjuvants, diluents and/or carriers; and (b) a pharmaceutical formulation including an amount of a second therapeutic compound selected from the group consisting of BH4, a pharmaceutically acceptable salt of BH4, a BH4 derivative and a pharmaceutically acceptable salt of a BH4 derivative, optionally in admixture with pharmaceutically acceptable adjuvants, diluents and/or carriers, wherein the first amount and the second amount together comprise a therapeutically effective amount for the prevention and/or treatment of respiratory diseases, and which components (a) and (b) are each provided in a form that is suitable for administration in conjunction with the other.

According to another aspect of the invention, there is provided a method of making a kit of parts as defined above, which method comprises bringing a component (a), as defined above, into association with a component (b), as defined above, thus rendering the two components suitable for administration in conjunction with each other.

Bringing the two components “into association with” each other, includes that components (a) and (b) of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

In case, the components (a) and (b) of the kit of parts are packaged and presented together as separate components of a “combination pack” for use in conjunction with each other in combination therapy the type of pharmaceutical formulation of the components (a) and (b) can be similar, for example, both components are formulated in separate tablets or capsules, or different, for example, one component is formulated as tablet or capsule and the other component is formulated for administration, for example, by inhalation.

Furthermore, there is provided a kit of parts comprising:

(I) one of components (a) or (b) as defined herein; together with
(II) instructions to use that component in conjunction with the other of the two components.

With respect to the kits of parts as described herein “administration in conjunction with” includes that respective formulations comprising a first therapeutic compound selected from the group consisting of Roflumilast, a pharmaceutically acceptable salt of Roflumilast, Roflumilast-N-oxide and a pharmaceutically acceptable salt of Roflumilast-N-oxide and a second therapeutic compound selected from the group consisting of BH4, a pharmaceutically acceptable salt of BH4, a BH4 derivative and a pharmaceutically acceptable salt of a BH4 derivative are administered sequentially, separately and/or simultaneously, over the course of treatment of the relevant disease.

Thus, in respect of the combination product according to the invention, the term “administration in conjunction with” includes that the two components of the combination product are administered (optionally repeatedly), either together (simultaneously), or sufficiently close in time (sequentially or separately), to enable a beneficial effect for the patient, that is greater, over the course of the treatment of the relevant disease, than if either a formulation comprising the first therapeutic agent, or a formulation comprising the second therapeutic agent, are administered (optionally repeatedly) alone, in the absence of the other component, over the same course of treatment, i.e. the administration of the two components of the combination product according to the invention results in a synergistic effect.

The synergistic effect(s) of the combination product(s) of the present invention encompass additional unexpected advantages for the prevention and/or treatment of respiratory diseases. Such additional advantages may include, but are not limited to, lowering the required dose of one or more of the therapeutic compounds of the combination products, reducing the side effects of one or more of the therapeutic compounds of the combination products or rendering one or more of the therapeutic compounds more tolerable to the patient in need of respiratory disease therapy.

The combined administration of Roflumilast, a pharmaceutically acceptable salt of Roflumilast, Roflumilast-N-oxide or a pharmaceutically acceptable salt of Roflumilast-N-oxide and BH4, a pharmaceutically acceptable salt of BH4, a BH4 derivative or a pharmaceutically acceptable salt of a BH4 derivative may also be useful for decreasing the number of separate dosages, thus, potentially improving compliance of the patient in need of respiratory disease therapy.

Further, in the context of a kit of parts according to the invention, the term “in conjunction with” includes that one or the other of the two components may be administered (optionally repeatedly) prior to, after, and/or at the same time as, administration of the other component.

A further aspect of the invention is the use of a combination product or kit of parts according to the invention for the manufacture of a pharmaceutical composition for the prevention and/or treatment of respiratory diseases.

Still a further aspect of the invention provides a method of prevention and/or treatment of respiratory diseases in a patient in need thereof which treatment or prophylaxis comprises administration to said patient a pharmaceutical formulation including an amount of a first therapeutic compound selected from the group consisting of Roflumilast, a pharmaceutically acceptable salt of Roflumilast, Roflumilast-N-oxide and a pharmaceutically acceptable salt of Roflumilast-N-Oxide, an amount of a second therapeutic compound selected from the group consisting of BH4, a pharmaceutically acceptable salt of BH4, a BH4 derivative and a pharmaceutically acceptable salt of a BH4 derivative, wherein the first amount and the second amount together comprise a therapeutically effective amount for the prevention and/or treatment of respiratory diseases, optionally in admixture with pharmaceutically acceptable adjuvants, diluents and/or carriers.

In another aspect of the present invention there is provided a method of prevention and/or treatment of respiratory diseases, which comprises administration of:
(a) a pharmaceutical formulation including an amount of a first therapeutic compound selected from the group consisting of Roflumilast, a pharmaceutically acceptable salt of Roflumilast, Roflumilast-N-oxide and a pharmaceutically acceptable salt of Roflumilast-N-oxide, optionally in admixture with pharmaceutically acceptable adjuvants, diluents and/or carriers; in conjunction with
(b) a pharmaceutical formulation including an amount of a second therapeutic compound selected from the group consisting of BH4, a pharmaceutically acceptable salt of BH4, a BH4 derivative and a pharmaceutically acceptable salt of a BH4 derivative, optionally in admixture with pharmaceutically acceptable adjuvants, diluents and/or carriers,
   wherein the first amount and the second amount together comprise a therapeutically effective amount for the prevention and/or treatment of respiratory diseases to a patient suffering from, or susceptible to, such a disease.

The term “therapeutically effective amount” as used herein refers to a characteristic of an amount of a therapeutic compound, or a characteristic of combined therapeutic compounds in combination therapy. The amount of combined amounts achieve the goal of preventing, avoiding, reducing or eliminating a disease or disorder.

The term “therapeutic compound” as used herein refers to a compound useful in the prevention and/or treatment of a disease or disorder.

Roflumilast is the international nonproprietary name (INN) for 3-cyclopropylmethoxy-4-difluoromethoxy-N-(3,5-dichloropyrid-4-yl)benzamide [structure of formula (1.1)]. The preparation of 3-cyclopropylmethoxy-4-difluoromethoxy-N-(3,5-dichloropyrid-4-yl)benzamide, its pharmaceutically acceptable salts and its N-oxide[3-cyclopropylmethoxy-4-difluoromethoxy-N-(3,5-dichloro-1-oxido-pyrid-4-yl)benzamide; structure of formula (1.2)] as well as the use of these compounds as phosphodiesterase (PDE) 4 inhibitors is described in the international patent application WO9501338.

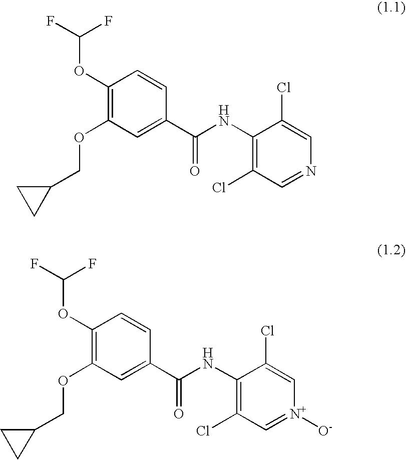

Salts encompassed within the term “pharmaceutically acceptable salts” of Roflumilast and Roflumilast-N-oxide refer to non-toxic salts of the said compounds; such salts are generally prepared by reacting a free base with a suitable organic or inorganic acid or by reacting an acid with a suitable organic or inorganic base. Particular mention may be made of the pharmaceutically acceptable inorganic and organic acids customarily used in pharmacy. Those suitable are in particular water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)-benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 1-hydroxy-2-naphthoic acid. As examples of pharmaceutically acceptable salts with bases may be mentioned, for example, the lithium, sodium, potassium, calcium, aluminium, magnesium, titanium, ammonium, meglumine or guanidinium salts.

“BH4” stands for (6R)-L-erythro-5,6,7,8-tetrahydrobiopterin (structure of formula 1.3):

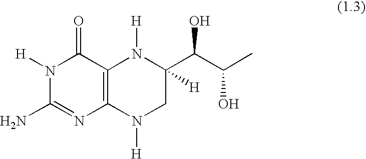

The term “BH4 derivative” as used herein refers to

(a) a compound of formula 1.4

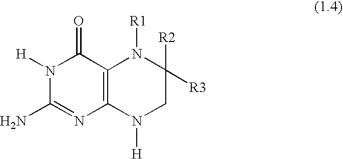

wherein R1 and R2 each represents a hydrogen atom or, taken together with each other, represent a single bond, while R3 represents —CH(OH)CH(OH)CH₃, —CH(OCOCH₃)CH(OCOCH₃)CH₃, —CH₃, —CH₂OH, or a phenyl group when R1 and R2 each represents a hydrogen atom, or —COCH(OH)CH₃ when R1 and R2 together represent a single bond, a stereoisomer or a pharmaceutically acceptable salt thereof, with the exception of (6R)-L-erythro-5,6,7,8-tetrahydrobiopterin.
or
(b) a compound of formula 1.5

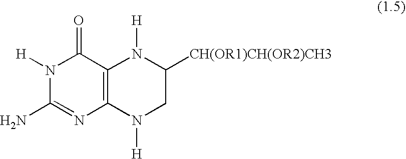

in which R1 and R2 independently from one another are an acyl group of general formula —C(O)R3, wherein R3 is hydrogen or a hydrocarbon residue having one or more carbon atoms, in particular 2 to 9 carbon atoms, or a pharmaceutically acceptable salt of this compound.

Preferable examples of the hydrocarbon residue represented by R3 are, for instance, a linear or branched alkyl group having one or more carbon atoms, preferably 2 to 9 carbon atoms, which is either saturated or unsaturated;

a substituted or unsubstituted phenyl group represented by the general formula

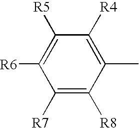

wherein R4, R5, R6, R7 and R8 are hydrogen or a linear or branched alkyl group in which the total of the carbon atoms thereof is preferably not more than 3;
a substituted or unsubstituted benzyl group represented by the general formula

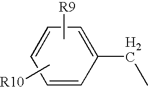

wherein R9 and R10 are hydrogen, methyl group or ethyl group in which the total of the carbon atoms thereof is preferably not more than 2;
and a substituted or unsubstituted arylalkyl group represented by the general formula

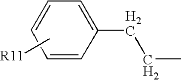

wherein R11 is hydrogen or a methyl group.

Among the above acyl groups —C(O)R3, the formyl group, acetyl group, propionyl group, butyryl group, isobutyryl group, valeryl group, isovaleryl group, and the benzoyl group are most preferable. It is also preferable that R1 and R2 are the same.

The compound of formula 1.5 has two diastereomers, i.e. 1′,2′-diacyl-(6R)-5,6,7,8-tetrahydro-L-biopterin and 1′,2′-diacyl-(6S)-5,6,7,8-tetrahydro-L-biopterin, which are diastereomeric at the 6-position. The term “BH4 derivatives” according to this invention includes each of the two diastereomers and a mixture thereof.

Preferred BH4 derivatives, which may be mentioned are

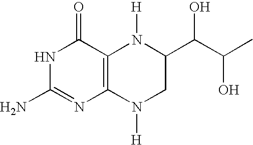

(6R,S)-5,6,7,8-tetrahydrobiopterin,

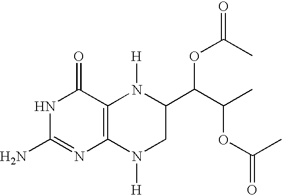

[1′,2′-diacetyl-5,6,7,8-tetrahydrobiopterin],

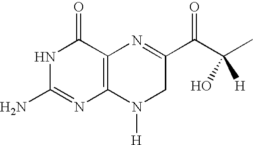

### [Sepiapterin],

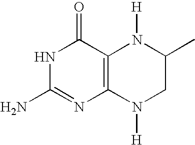

[6-methyl-5,6,7,8-tetrahydropterin],

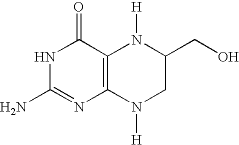

[6-hydroxymethyl-5,6,7,8-tetrahydropterin],

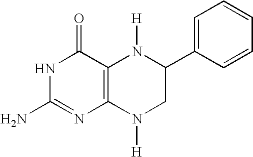

[6-phenyl-5,6,7,8-tetrahydropterin],
and the pharmaceutically acceptable salts of these compounds.

As pharmaceutically acceptable salts of BH4 and BH4 derivatives may be mentioned, by way of example, the salts of these compounds with pharmacologically non-toxic acids, including mineral acids such as hydrochloric acid, phosphoric acid, sulfuric acid, boric acid; and organic acids such as acetic acid, formic acid, maleic acid, fumaric acid and mesylic acid.

In a preferred embodiment the pharmaceutically acceptable salt of BH4 is the dihydrochloride salt of BH4.

It is understood that the therapeutic compounds and their pharmaceutically acceptable salts mentioned can also be present, for example, in the form of their pharmaceutically acceptable solvates, in particular in the form of their hydrates.

The term “respiratory diseases” according to this invention refers to pulmonary diseases with an underlying partial and global respiratory failure, i.e. with an impairment of oxygen uptake or carbon dioxide release in the lung. In the healthy lung of humans both at rest and during exercise there are always areas of good and poor or absolutely no ventilation existing simultaneously side by side (ventilation inhomogeneity). An as yet unknown mechanism ensures that there is little or no perfusion of the capillaries adjacent to alveoli with little or no ventilation. This occurs in order to minimize inefficient perfusion of areas of the lung which are not involved in gas exchange. During bodily exercise, the distribution of ventilation changes (recruitment of new alveoli) and there is increased perfusion of the relevant capillary bed. Conversely, when there is less ventilation due to physiological or pathological processes (airway obstruction), the capillary flow are reduced through vasoconstriction. This process is referred to as hypoxic vasoconstriction (Euler-Liljestrand mechanism). When this adaptation mechanism of ventilation and perfusion is impaired (“mismatch”), there may, despite adequate ventilation and normal perfusion of the lungs, be a more or less pronounced collapse of the gas exchange function, which can be compensated only inadequately despite a further increase in ventilation or perfusion. Under these conditions there are regions, which are not ventilated but are well perfused (shunting) and those which are well ventilated but not perfused (dead space ventilation). The consequences of this “ventilation/perfusion mismatch” are hypoxemia (deterioration in gas exchange with decrease in the oxygen content of the patient's blood), wasted perfusion (uneconomical perfusion of unventilated areas) and wasted ventilation (uneconomical ventilation of poorly perfused areas).

The cause of this “partial and global respiratory failure” is inadequate adaptation of the intrapulmonary perfusion conditions to the inhomogeneous pattern of the distribution of ventilation. The resulting mismatch derives from the effect of vasotherapeutic (inflammatory) mediators which prevail over the physiological adaptation mechanism. This effect is particularly evident during exercise and when the oxygen demand is increased and it is manifested by dyspnoea (hypoxia) and limitation of body performance.

The term “partial respiratory failure” according to the invention relates to a fall in the O₂ partial pressure in the blood as a manifestation of the aforementioned impairment of oxygen uptake or carbon dioxide release.

The term, “global respiratory failure” according to the invention relates to a fall in the O₂ partial pressure in the blood and a rise in the CO₂ partial pressure in the blood as a manifestation of the aforementioned impairment of oxygen uptake or carbon dioxide release.

In patients with inflammatory and degenerative lung disorders such as, for example, chronic obstructive pulmonary disease (COPD), bronchial asthma, pulmonary hypertension, pulmonary fibroses, emphysema, interstitial pulmonary disorders and pneumonias, partial or global respiratory failure is observed. Thus, according to this invention, the term “respiratory diseases” or “respiratory diseases with an underlying partial or global respiratory failure” refers to one or more of the following clinical conditions: COPD, bronchial asthma, pulmonary hypertension, pulmonary fibroses, emphysema, interstitial pulmonary disorders or pneumonias.

The term “COPD” is the abbreviation for chronic obstructive pulmonary disease. Patients suffering from COPD are characterized by pulmonary alterations as well as extra-pulmonary alterations such as limited body performance. Pulmonary alterations are changes of airways obstructed due to inflammation, mucus hypersecretion and changes of pulmonary vessels. The resulting limited airflow and the loss of respiratory epithelium results in impaired oxygenation. In addition, pulmonary blood circulation is impaired due to vascular remodeling [Santos S et al. Eur Respir J 2002 19: 632-8] and due to a ventilation/perfusion mismatch deriving from the effect of vasoactive (inflammatory) mediators prevailing over the physiological adaptation mechanism and in part from structural changes of the lung capillaries which develop during the disease progression. This effect is particularly evident during exercise and when the oxygen demand is increased and it is manifested by dyspnoea (hypoxia) and limitation of body performance.

The invention is based on the theory that a combination of the PDE4 inhibitor Roflumilast with BH4 is suitable for the treatment of patients with partial and global respiratory failure. Inhibitors of phosphodiesterase 4 have been shown to block superoxide production from NADPH oxidases in inflammation as it occurs in respiratory diseases. Increased superoxide production in the vasculature has been shown to reduce the active BH4 concentration preferentially (Kuzkaya et al; J Biol Chem 2003 278, 22546-54; Landmesser et al. J Clin Invest 2003, 111, 1201-9). According to this invention, in the endothelium, dysregulation of NADPH oxidases and NO synthases and the increase of ONOO⁻ concentration lead to oxidation of BH4 and thus to reduced BH4 concentration in the lungs and in skeletal muscle. Reduced BH4 concentrations result in uncoupling of NOS (iNOS and eNOS) and in an increase in superoxide concentration and finally in the production of ONOO⁻. An increase in superoxide anion concentration leads to more ONOO⁻ and the resulting increase in ONOO⁻ leads to less BH4 in the lungs and in the skeletal muscle. This circle of superoxide and ONOO⁻ production as well as BH4 inactivation finally results in endothelial dysfunction and in a ventilation/perfusion mismatch. The administration of a combination of the PDE4 inhibitor Roflumilast and BH4 leads to a reduced generation of superoxide anions and ONOO⁻ and consequentially to a re-coupling of NOS (i.e. NOS produce NO instead of superoxide anions) and an increase in bioavailable NO which inter alia results in vasodilation and the reduction of the ventilation/perfusion mismatch.

The term “prevention and/or treatment of respiratory diseases” as well as “prevention and/or treatment of respiratory diseases with an underlying partial or global respiratory failure” and therewith the term “prevention and/or treatment of COPD” refers to the circumstance that the administration of a combination of the PDE4 inhibitor Roflumilast with BH4 leads to dilatation of vessels in the pulmonary circulation and, at the same time, to a redistribution of the blood flow within the lung in favor of the well-ventilated areas. This principle, referred to hereinafter as rematching, leads to an improvement in the gas exchange function both at rest and during physical exercise in the lungs in patients suffering from partial or global respiratory failure, such as COPD patients. Rematching does not only result in an improved gas exchange in the lungs but also in improved gas exchange in skeletal muscles and therefore in an improvement of physical performance. The term “prevention and/or treatment of muscular dysfunction in COPD patients” exactly refers to this positive outcome of the administration of a combination of the PDE4 inhibitor Roflumilast with BH4 in COPD patients.

The therapeutic compounds of the present invention can be administered by any appropriate route known to the person skilled in the art. The formulations include those suitable for oral, parenteral (including subcutaneous, intradermal, intramuscular, intravenous and intraarticular), intranasal, inhalation (including fine particle dusts or mists which may be generated by means of various types of metered dose pressurized aerosols, nebulisers or insufflators), rectal and topical (including dermal, buccal, sub-lingual and intraocular) administration although the most suitable route may depend upon for example the condition and disorder of the recipient.

The therapeutic compounds of the present invention can be administered by a variety of methods known in the art, although for many therapeutic applications, the preferred route of administration is the oral route. Another preferred route of administration is by way of inhalation.

In case of pharmaceutical compositions, which are intended for oral administration, the therapeutic compounds are formulated to give medicaments according to processes known per se and familiar to the person skilled in the art. The therapeutic compound(s) are employed as medicament, preferably in combination with suitable pharmaceutical carrier(s), adjuvant(s) and/or diluent(s), in the form of tablets, coated tablets, capsules, emulsions, suspensions, syrups or solutions, the therapeutic compound(s) content advantageously being between 0.1 and 95% by weight and, by the appropriate choice of the carrier, it being possible to achieve a pharmaceutical administration form precisely tailored to the therapeutic compound(s) and/or to the desired onset of action (e.g. a sustained-release form or an enteric form).

The person skilled in the art is familiar on the basis of his/her expert knowledge which carrier(s), adjuvant(s) or diluent(s) are suitable for the desired pharmaceutical formulations. In addition to solvents, gel-forming agents, tablet excipients and other therapeutic compound carriers, it is possible to use, for example, antioxidants, dispersants, emulsifiers, antifoams, flavor corrigents, preservatives, solubilizers, colorants or permeation promoters and complexing agents (e.g. cyclodextrins).

Pharmaceutical formulations, which comprise BH4 preferably contain as an adjuvant an antioxidant, such as, for example ascorbic acid. Further adjuvants, which are beneficial in pharmaceutical formulations which comprise BH4 are L-cysteine or N-acetyl-L-cysteine.

Formulations for inhalation include powder compositions, which will preferably contain lactose, and spray compositions which may be formulated, for example, as aqueous solutions or suspensions or as aerosols delivered from pressurized packs, with the use of a suitable propellant, e.g. 1,1,1,2-terafluorethane, 1,1,1,2,3,3,3-heptafluoropropane, carbon dioxide or other suitable gas. A class of propellants, which are believed to have minimal ozone-depleting effects in comparison to conventional chlorofluorocarbons comprise hydrofluorocarbons and a number of medicinal aerosol formulations using such propellant systems are disclosed in, for example, EP0372777, WO9104011, WO9111173, WO9111495, WO9114422, WO9311743, and EP0553298. These applications are all concerned with the preparation of pressurized aerosols for the administration of medicaments and seek to overcome problems associated with the use of this new class of propellants, in particular the problems of stability associated with the pharmaceutical formulations prepared. The applications propose, for example, the addition of one or more of excipients such as polar cosolvents (e.g. alcohols such as ethanol), alkanes, dimethyl ether, surfactants (including fluorinated and non-fluorinated surfactants, carboxylic acids such as oleic acid, polyethoxylates etc.) or bulking agents such as a sugar (see for example WO0230394). For suspension aerosols, the therapeutic compounds should be micronised so as to permit inhalation of substantially all of the therapeutic compounds into the lungs upon administration of the aerosol formulation, thus the therapeutic compounds will have a particle size of less than 100 microns, desirably less than 20 microns, and preferably in the range 1 to 10 microns, for example, 1 to 5 microns.

The exact dosage and regimen for administering the combination according to the invention will necessarily depend upon the potency, the duration of action of the therapeutic compounds used, the nature and severity of the disease to be treated, as well as the sex, age, weight, general health and individual responsiveness of the patient to be treated, and other relevant circumstances.

While not intended to be limiting, in case of oral administration of a BH4 preparation, it has proven advantageous to administer 1 to 3 tablets of the preparation per day whereby one tablet contains 10 to 500 mg of BH4 or a BH4 derivative.

Preferably, the preparations according to the invention are administered per application in such an amount that the amount of BH4 or BH4 derivative is between 0.2 and 50 mg per kilogram of body weight per day. As a rule in the long term treatment of chronic respiratory diseases, such as COPD, BH4 or the BH4 derivative may be administered 1 to 3 times per day in a dosage of 10-500 mg over a period of several years. In the treatment of acute episodes of chronic disorders it may be possible to increase the daily dosage of BH4 or the BH4 derivative up to 2000 mg.

Continuous treatment of chronic respiratory diseases may also be accomplished by administering BH4 or a BH4 derivative by inhalation, or by intravenous or subcutaneous administration.

In the case of inhalative administration of BH4 or a BH4 derivative, BH4 or the BH4 derivative are formulated in a form known to the person skilled in the art and dosed in an order of magnitude customary for a person in need of the treatment. It has been proven advantageous to administer BH4 by inhalation in the following application scheme: Preferably, 10 to 1000 mg BH4 are dissolved in sterile water containing 1% ascorbic acid. The solution is administered using an inhalation device 1 to 3 times per day in such an amount that the final amount of BH4 is between 0.2 and 50 mg per kilogram of body weight per day. It has been proven advantageous to continuously administer BH4 by inhalation 1 to 3 times in a dosage of 10 to 1000 mg per day. In the treatment of acute episodes of chronic disorders it may be possible to increase the dosage in accordance with the experience of the attending physician.

In case of oral administration of 3-cyclopropylmethoxy-4-difluoromethoxy-N-(3,5-dichloropyrid-4-yl)benzamide (Roflumilast), the adult daily dose is in the range from 50-1000 μg, preferably in the range from 50-500 μg, more preferably in the range from 250-500 μg, preferably by once daily administration.

Suitable oral dosage forms of Roflumilast and Roflumilast-N-oxide are described in the international patent application WO03070279.

In case of intravenous administration of 3-cyclopropylmethoxy-4-difluoromethoxy-N-(3,5-dichloropyrid-4-yl)benzamide (Roflumilast), the adult daily dose is in the range from 50-500 μg, preferably in the range from 150-300 μg.

Suitable dosage forms of Roflumilast and Roflumilast-N-oxide for i. v. administration are described in the international patent application WO2006032676.

In one preferred embodiment, Roflumilast and BH4 are administered simultaneous in two different oral pharmaceutical compositions.

In another preferred embodiment, Roflumilast and BH4 are administered more or less simultaneous but separately via different routes. In this preferred embodiment, BH4 is administered by inhalation and Roflumilast is administered orally.

In still another preferred embodiment, Roflumilast and BH4 are administered together in one oral pharmaceutical composition.

In a further preferred embodiment, Roflumilast and BH4 are administered more or less simultaneous but separately via different routes. In this further preferred embodiment, BH4 is administered orally and Roflumilast is administered by inhalation.

### EXAMPLES

### Example 1

### Production of an Injectable BH4 Preparation

To make up a homogenous solution 1.5 g BH4 dihydrochloride, 1.5 g Ascorbic acid, 0.5 g L-cystein hydrochloride and 6.5 g mannitol are dissolved into sterile purified water to make up 100 ml, then sterilized, 1 ml aliquot each is dispensed into a vial or ampule, lyophilized and sealed.

### Example 2

### Production of an Injectable BH4 Preparation

Under anaerobic atmosphere 2.0 g of BH4 dihydrochloride is dissolved in sterile deionized water to make up 100 ml, the sterilized and sealed.

### Example 3

### Production of a BH4 Tablet Preparation

Ten parts of ascorbic acid and 5 parts of L-cysteine hydrochloride are added to 1 part of polyvinylpyrrolidone which is dissolved in sterilized deionised water before to give a homogenous solution. Then, 10 parts of BH4 dihydrochloride are added to prepare a homogenous solution. This solution is mixed with 58 parts of lactose and 15 parts of microcrystalline cellulose and 1 part of magnesium stearate and tableted.

### Example 4

Production of a Roflumilast Tablet Preparation

### a) Weight Based on a Tablet Containing 0.125 mg of Roflumilast

| | | |
|---|---|---|
| 1. | Roflumilast | 0.125 mg |
| 2. | Lactose monohydrate | 49.660 mg |
| 3. | Corn starch | 13.390 mg |
| 4. | Polyvidone K90 | 1.300 mg |
| 5. | Magnesium stearate (vegetable) | 0.650 mg |
| | Total | 65.125 mg |

Production: (1) is mixed with part of (3), and a trituration is produced in a planetary mill. The trituration is put together with (2) and the remaining amount of (3) in the product container of a fluidized bed granulation system, and a 5% granulation solution of (4) in purified water is sprayed on and dried under suitable conditions. (5) is added to the granules, and the mixture obtained after mixing is compressed in a tablet press to tablets having an average weight of 65.125 mg.

### b) Weight Based on a Tablet Containing 0.25 mg of Roflumilast

| | | |
|---|---|---|
| 1. | Roflumilast | 0.250 mg |
| 2. | Microcrystalline cellulose | 33.900 mg |
| 3. | Corn starch | 2.500 mg |
| 4. | Polyvidone K90 | 2.250 mg |
| 5. | Sodium carboxymethylstarch (type A) | 20.000 mg |
| 6. | Magnesium stearate (vegetable) | 0.600 mg |
| | Total | 59.500 mg |

Production: (1) is mixed with part of (3), and a trituration is produced in a planetary mill. The trituration is put together with (2), (5) and the remaining amount of (3) in the product container of a fluidized bed granulation system, and a 5% granulation solution of (4) in purified water is sprayed on and dried under suitable conditions. (6) is added to the granules, and the mixture obtained after mixing is compressed in a tablet press to tablets having an average weight of 59.5 mg.

c) Weight Based on a Tablet Containing 0.5 mg of Roflumilast

| | | |
|---|---|---|
| 1. | Roflumilast (micronized) | 0.500 mg |
| 2. | Lactose monohydrate | 49.660 mg |
| 3. | Corn starch | 13.390 mg |
| 4. | Polyvidone K90 | 1.300 mg |
| 5. | Magnesium stearate (vegetable) | 0.650 mg |
| | Total | 65.500 mg |

Production: A 5% granulation solution of (4) in purified water is produced. (1) is suspended into the solution. (2) and (3) are filled into the product container of a fluidized bed granulation system. The suspension is sprayed on and dried under suitable conditions. (5) is added to the granules, and the mixture obtained after mixing is compressed in a tablet press to tablets having an average weight of 65.5 mg.

1. A combination product which comprises a pharmaceutical formulation including an amount of a first therapeutic compound selected from the group consisting of Roflumilast, a pharmaceutically acceptable salt of Roflumilast, Roflumilast-N-oxide and a pharmaceutically-acceptable salt of Roflumilast-N-oxide, an amount of a second therapeutic compound selected from the group consisting of BH4, a pharmaceutically acceptable salt of BH4, a BH4 derivative and a pharmaceutically acceptable salt of a BH4 derivative, wherein the first amount and the second amount together comprise a therapeutically effective amount for the prevention and/or treatment of respiratory diseases, and optionally pharmaceutically-acceptable adjuvants, diluents and/or carriers.

2. A combination product comprising: (A) an amount of a first therapeutic compound selected from the group consisting of Roflumilast, a pharmaceutically acceptable salt of Roflumilast, Roflumilast-N-oxide and a pharmaceutically-acceptable salt of Roflumilast-N-oxide; and (B) an amount of a second therapeutic compound selected from the group consisting of BH4, a pharmaceutically acceptable salt of BH4, a BH4 derivative and a pharmaceutically acceptable salt of a BH4 derivative, wherein the first amount and the second amount together comprise a therapeutically effective amount for the prevention and/or treatment of respiratory diseases and wherein each of components (A) and (B) is optionally formulated in admixture with pharmaceutically-acceptable adjuvants, diluents and/or carriers.

3. The combination product as claimed in claim 2 which comprises a kit of parts comprising components: (a) a pharmaceutical formulation including an amount of a first therapeutic compound selected from the group consisting of Roflumilast, a pharmaceutically acceptable salt of Roflumilast, Roflumilast-N-oxide and a pharmaceutically-acceptable salt of Roflumilast-N-oxide, optionally in admixture with pharmaceutically-acceptable adjuvants, diluents and/or carriers; and (b) a pharmaceutical formulation including an amount of a second therapeutic compound selected from the group consisting of BH4, a pharmaceutically acceptable salt of BH4, a BH4 derivative and a pharmaceutically acceptable salt of a BH4 derivative, optionally in admixture with pharmaceutically-acceptable adjuvants, diluents and/or carriers, wherein the first amount and the second amount together comprise a therapeutically effective amount for the prevention and/or treatment of respiratory diseases and which components (a) and (b) are each provided in a form that is suitable for administration in conjunction with the other.

4. The combination product as claimed in claim 3, wherein, in the kit of parts, components (a) and (b) are suitable for sequential, separate and/or simultaneous use in the prevention and/or treatment of respiratory diseases.

5. The combination product as claimed in claim 1, wherein the first therapeutic compound is Roflumilast.

6. The combination product as claimed in claim 1, wherein the first therapeutic compound is Roflumilast-N-oxide.

7. The combination product as claimed in claim 1, wherein the second therapeutic compound is (6R)-L-erythro-5,6,7,8-tetrahydrobiopterin.

8. The combination product as claimed in claim 1, wherein the second therapeutic compound is (6R)-L-erythro-5,6,7,8-tetrahydrobiopterin dihydrochloride.

9. The combination product as claimed in claim 1, wherein the second therapeutic compound is selected from the group consisting of (6R,S)-5,6,7,8-tetrahydrobiopterin, 1′,2′-diacetyl-5,6,7,8-tetrahydrobiopterin, sepiapterin, 6-methyl-5,6,7,8-tetrahydropterin, 6-hydroxymethyl-5,6,7,8-tetrahydropterin, 6-phenyl-5,6,7,8-tetrahydropterin and a pharmaceutically acceptable salt of these compounds.

10. The combination product as claimed in claim 1, wherein the second therapeutic compound is sepiapterin or a pharmaceutically acceptable salt thereof.

11. A process for the preparation of a kit of parts, which process comprises bringing a component (a), as defined in claim 3, into association with a component (b), as defined in claim 3, thus rendering the two components suitable for administration in conjunction with each other.

12. A kit of parts comprising:
(I) one of components (a) and (b) as defined in claim 3, together with (II) instructions to use that component in conjunction with the other of the two components.

13-15. (canceled)

16. A method for the prevention and/or treatment of one or more respiratory diseases, which comprises administration of a combination product as defined in claim 1 to a patient suffering from, or susceptible to, such a disease.

17. The method as claimed in claim 16, wherein the respiratory disease is selected from the group consisting of COPD, bronchial asthma, pulmonary hypertension, pulmonary fibrosis, emphysema, interstitial pulmonary disorders, and pneumonias.

18. The method as claimed in claim 17, wherein the respiratory disease is COPD.

19. A method for the prevention and/or treatment of muscular dysfunction in COPD patients, which comprises administration of a combination product as defined in claim 1 to a patient suffering from, or susceptible to, such a disease.

20-22. (canceled)
